(19) United States
(12) Patent Application Publication
(10) Pub. No.: US 2018/0264161 A1
(43) Pub. Date: Sep. 20, 2018

(54) Ultraviolet Light Germicidal Facemask Apparatus and Method

(71) Applicant: Gerry M. Welch, Cooperstown, NY (US)

(72) Inventor: Gerry M. Welch, Cooperstown, NY (US)

(21) Appl. No.: 15/977,300

(22) Filed: May 11, 2018

(51) Int. Cl. A61L 9/20; A61M 16/06

(52) U.S. Cl. CPC A61L 9/20; B01D 2257/91; A61M 16/06

(57) ABSTRACT

A germicidal facemask apparatus and related method, comprising: an inner facemask shell comprising at least one inner shell vent thereof; an outer facemask shell shaped to substantially conform to the inner facemask shell; a substantially-airtight attachment of the outer shell with the inner shell about perimeters thereof; a substantially airtight and light-reflective air cavity between the inner and outer facemask shells, formed by the substantially airtight attachment of the outer shell with the inner shell about the perimeters, and by inner shell and outer shell substantially-reflective materials; and an ultraviolet light source configured to introduce ultraviolet light into the air cavity between the inner and outer facemask shells and for the ultraviolet light to substantially permeate the air cavity by virtue of the substantially-reflective materials on the front surface of the inner facemask shell and on the rear surface of the outer facemask shell.

### BACKGROUND OF THE INVENTION

Facemasks for air filtration are commonly used in hospitals and clinics, and in other places where it is desirable to prevent the spread of airborne diseases transmitted through the human respiratory system. These masks generally comprise a tightly-woven material with micropores which allow air to pass through the mask when the wearer/user is breathing in and out. These micropores are small enough to bar the passage of most pathogens. Yet, there are pathogens which can still pass through these masks and as such, the effectiveness of these masks is still somewhat limited. This includes pathogens which can pass through the edges of the mask due to the pressures causes by breathing through the microporous material. As such, in a world where Eboli, influenza, strep and even the common cold can be cause for concern, improvements to these prior art facemasks are warranted.

### SUMMARY OF THE INVENTION

Disclosed herein is a germicidal facemask apparatus and related method, comprising: an inner facemask shell shaped to fit in a substantially airtight fashion over a mouth and nose of a human face; an outer facemask shell shaped to substantially conform to the inner facemask shell; the inner facemask shell comprising at least one inner shell vent thereof; the outer facemask shell comprising at least one outer shell vent thereof; a front surface of the inner facemask shell comprising an inner shell substantially-reflective material thereof; a rear surface of the outer facemask shell comprising an outer shell substantially-reflective material thereof; a substantially-airtight attachment of the outer shell with the inner shell about perimeters thereof; a substantially airtight and light-reflective air cavity between the inner and outer facemask shells, formed by the substantially airtight attachment of the outer shell with the inner shell about the perimeters, and by the inner shell and outer shell substantially-reflective materials; and an ultraviolet light source configured to introduce ultraviolet light into the air cavity between the inner and outer facemask shells and for the ultraviolet light to substantially permeate the air cavity by virtue of the substantially-reflective materials on the front surface of the inner facemask shell and on the rear surface of the outer facemask shell; wherein when the germicidal facemask apparatus is placed over a user's face, by virtue of the foregoing configuration combination, air which is breathed in and out by the user will pass through the air cavity while traversing between the at least one inner shell vent and the at least one outer shell vent, and will undergo germicidal sterilization responsive to its being exposed to the ultraviolet light being reflected within the cavity.

### BRIEF DESCRIPTION OF THE DRAWINGS

The features of the invention believed to be novel are set forth in the appended claims. The invention, however, together with further objects and advantages thereof, may best be understood by reference to the following description taken in conjunction with the accompanying drawing(s) summarized below.

FIG. 1 illustrates a front plan view of an outer facemask shell employed in accordance with the invention.

FIG. 2 illustrates a front plan view of an inner facemask shell employed in accordance with the invention.

FIG. 3 illustrates a front plan view of the inner facemask shell attached behind the outer facemask shell to form the complete ultraviolet light germicidal facemask of the invention.

### DETAILED DESCRIPTION

In accordance with a preferred embodiment of the invention, two shells of soft but firm lightweight material are joined together with an intermediate air cavity/pocket into which ultraviolet light is projected, to form a germicidal facemask. Each individual shell is similar in shape to the filtration facemasks used in the prior art insofar as these shells are configured to conform to the face of a user, but with the novel and inventive differences that will now be disclosed.

Both shells comprise a lightweight material. The surfaces of each shell facing inward toward the air cavity comprise a substantially reflective material. Ultraviolet light is then introduced between the two shells. The reflective nature of the inner walls of the air cavity bathes the air cavity, and thus air that is breathed through the air cavity, with germicidal ultraviolet light. Air is allowed to easily access and egress through shells. This is accomplished by air passages containing louvered vents which allow air to pass but substantially bar light passage. The vents are made of soft flexible plastic or equivalent material and are fastened to the shells by glue, snaps, or any equivalent attachment. One or more louvered vents is attached to the outer shell, preferably opposite where the user's mouth is to be located. One or more (preferably two) louvered vents are attached, preferably, to the upper portion of the inner shell, and in any event, preferably at a different location than the vents are attached to the outer shell. The ultraviolet light kills pathogens that enter the chamber upon the user breathing both in and out. The foregoing will now be illustrated in more detail by reference to the drawings.

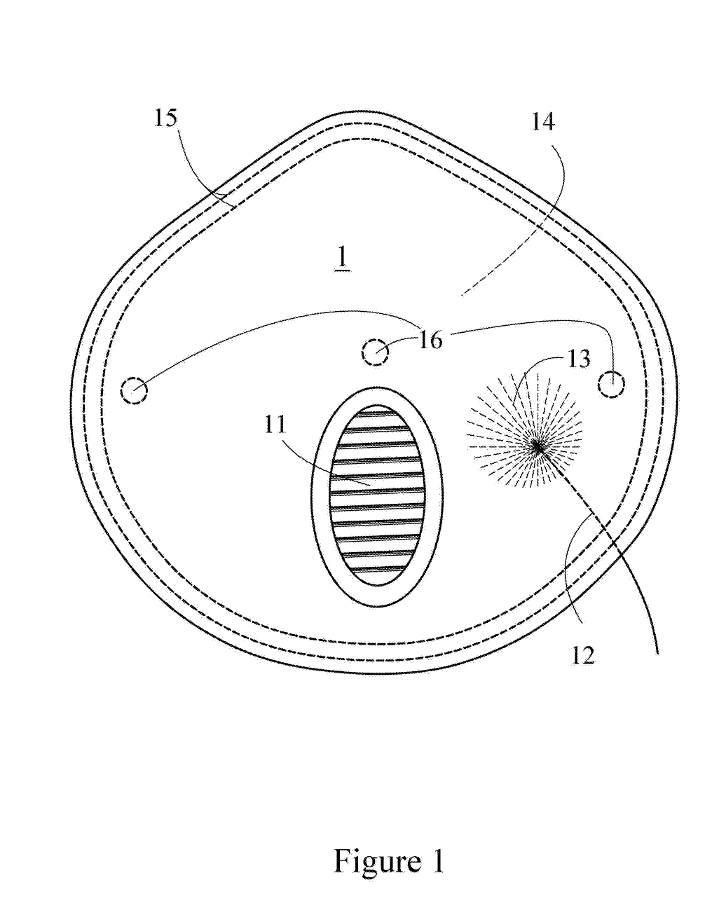

FIG. 1 illustrates an outer facemask shell 1 employed in accordance with the invention. This outer shell 1 comprises at least one outer shell vent 11 which is preferably louvered. As shown in this FIG. 1, this outer shell vent 11 is preferably situated in front of where the user's mouth will be, but this is for example, not limitation. The benefit of this placement is visual, insofar as someone viewing the mask while another person is wearing the mask will see the vent in front of the user's mouth, rather than in front of some other part of their face.

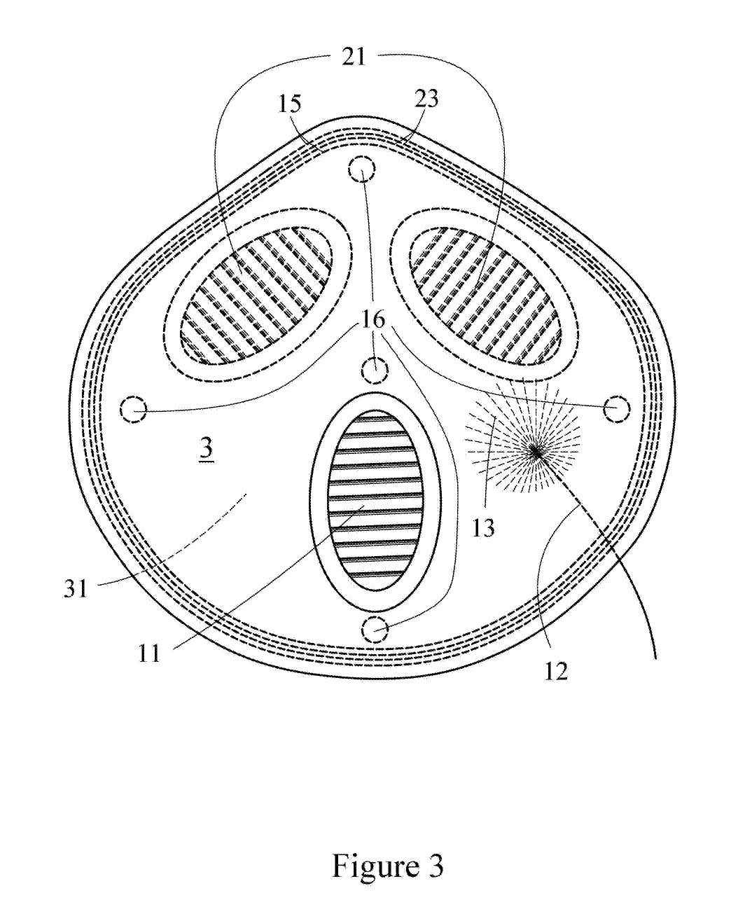

A germicidal ultraviolet light source 12 such as but not limited to an optical fiber is used to introduce ultraviolet light behind this outer shell 1, as illustrated by the hidden lines shown for the optical fiber in FIG. 1 once the fiber enters behind the shell 1. The ultraviolet light 13 introduced by the light source 12 is also illustrated by hidden lines to indicate that this light exits the source 12 from behind the outer shell 1. The rear surface of the outer shell 1 which is hidden from view in FIG. 1 comprises a substantially reflective outer shell coating 14 the purpose of which together with a similar coating on the front surface of the inner shell 2 facilitates ultraviolet light propagation throughout an air cavity 31 that is formed when the two shells are mated together, as will be discussed in relation to FIG. 3.

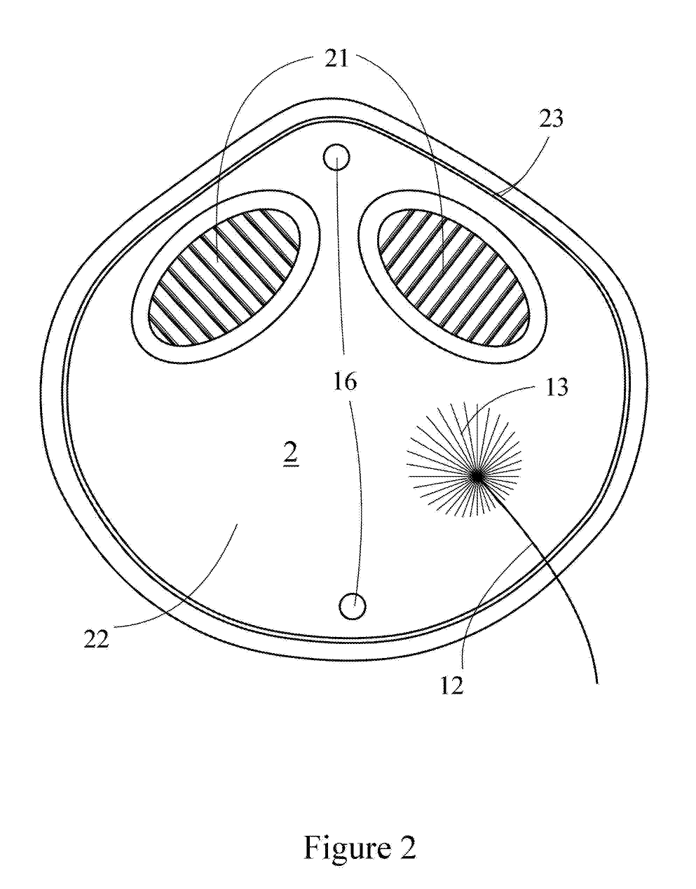

Further, outer shell 1 comprises an outer shell attachment 15 about a perimeter thereof, configured to mate with an inner shell attachment 23 illustrated in FIG. 2, so that the two shells can be attached and mated together in an airtight fashion to confine the air between the shells being subjected to the ultraviolet light. Preferably, this attachment 15, 23 enables the shells to be separated for cleaning and repairs, but it is also envisioned with the scope of this disclosure and the claims that this attachment might be fabricated to be permanent so that the shells cannot be separated. Finally, the inner surface of outer shell 1 may comprise one or more small spacers 16, which may be simple protruding nubs, which are used to provide the requisite spacing to facilitate proper breathing airflow and facilitate light penetration after the outer 1 and inner 2 shells have been mated together as described in relation to FIG. 3.

FIG. 2 illustrates an inner facemask shell 2 employed in accordance with the invention. This inner shell 2 comprises at least one inner shell vent 21 which is preferably louvered. FIG. 2, for example not limitation, shows two such louvered vents 21. These vents 21 are illustrated near the top part of the inner shell 2 on either side of where the user's nose will be situated, but this placement is also for example, not limitation. The benefit of placing these vents on either side of the nose rather than somewhere else is that this is more comfortable for the user. But it is particularly desirable that if the outer shell vent or vents 11 are placed on the lower portion of the outer shell 1 as illustrated, the inner shell vent or vents 21 be placed on the upper portions of the shell, and vice versa, because this will force the air passing through an air cavity 31 between the two shells to traverse a longer distance while it is in the cavity, thus maximizing the air's exposure to the ultraviolet light 13.

In FIG. 2 the ultraviolet light 13 and its source 12 are shown in solid lines, because from this view these are in front of the inner shell 2, and indeed, are between the two shells once the shells are mated together. The front surface of outer shell 2 which is visible from the view of FIG. 2 comprises a substantially reflective inner shell coating 22 similar in character to the reflective coating 14 on the rear surface of the outer shell 1. When the shells are put together as will momentarily be described, this maximizes the exposure to the ultraviolet light 22 of air passing through the air cavity 31 between the two shells.

Also, inner shell 2 comprises an inner shell attachment 23 about a perimeter thereof, configured to mate with the outer shell attachment 15 of FIG. 1, again, so that the two shells can be attached and mated together in an airtight fashion to confine the air between the shells and subject that air to the ultraviolet light 13. Finally, the outer surface of the inner shell 2, just like the inner surface of the outer shell 1, may comprise one or more small spacers 16 to provide the requisite spacing to permit proper airflow and facilitate light penetration, after the outer 1 and inner 2 shells have been mated together as will be described in reference to FIG. 3.

FIG. 3 now illustrates the mating of the inner shell 1 with the outer shell 2 to form the complete germicidal facemask 3 of the invention. Everything that is not directly visible from in front of the facemask 3 is illustrated by hidden lines. Specifically, the outer shell attachment 15 and the inner shell attachment 23 are simple mated together as shown to form a substantially airtight seal, thereby creating an air cavity 31 between the inner 1 and outer 2 shells. Then, when the entire germicidal facemask 3 is placed over the user's face and the ultraviolet light 13 is fed into the cavity 31 between the shells by the ultraviolet light source 12, air that is breathed in or out by the user will be exposed to the ultraviolet light and thereby undergo germicidal sterilization.

In the present illustration of FIG. 3, for example not limitation, the outer 15 and inner 23 shell attachments simply comprise the male and female portions (or vice versa) of a so-called Ziploc® seal, which as known in the art uses a two-part strip along the opening of a plastic bag whereby when pressed together the bag seals the contents inside from air exposure. Other possibilities for the matable attachment of the two shells may include Velcro® and the like, or even simple snaps. The use of a Ziploc® attachment or equivalent is preferred, however, because these are specifically configured to provide an airtight seal which is most desirable for the air cavity 31 between the two shells 1 and 2.

It is very desirable that the material used for the outer 1 and inner 2 shells be flexible to provide for user comfort and fit and to accommodate variable facial characteristics, but also that this material be air-impermeable so as to not allow the passage of air other than through the vents 11 and 21. Additionally, this provides the best surfaces between the shells for the substantially reflective coatings 14 and 22 shown in FIGS. 1 and 2.

In FIG. 3, it will be appreciated by observing the differing juxtapositions of the outer shell vent(s) 11 with the inner shell vent(s) 21 horizontally and vertically relative to a geometric plane defined by the mask 3, that air passing through the cavity 31 will be forced by virtue of shell material being air-impermeable other than through the vents 11 and 21, to traverse a larger space inside of the cavity 31 before it either passes out the front of the mask 3 or into the user's nose and mouth through the rear of the mask 3. As a result, the exposure of the air to the ultraviolet light for is prolonged. Specifically, it will be seen how the outer shell vent 11 aligns with an air-impermeable portion of the inner shell 2 while the inner shell vents 21 align with an air-impermeable portion of the outer shell.

It has also been stated that the vents 11 and 21 are preferably louvered. The purpose of the louvers is simply to prevent the ultraviolet light 13 from escaping the cavity 31, and by their angles, to better facilitate airflow inside the cavity 31. In order to best prevent the ultraviolet light 13 from escaping, the louvers may be curved with a suitably-designed surface, and/or some form of baffles may be employed so long that the airflow for the user's breathing is not overly restricted.

Due to the curvatures of the overall shells 1 and 2 to accommodate the user's face, and to the inner shell being closer to the face than the outer shell, the inner shell 2 may be fabricated to be slightly smaller than the outer shell 1 so they may be snugly fitted together. The vents 11 and 21 have been illustrated as ovals, but this is for example not limitation. It is expected that then the two shells are pressed together there will be an approximate ¼″ separation, up to perhaps ½″, defining the air cavity 31. It will now be appreciated how the spacers 16 already shown in FIGS. 1 and 2, when the two shells 1 and 2 are mated together, will to enforce the requisite spacing between the shell to maintain the cavity 31 to permit proper airflow and allow the ultraviolet light to propagate widely throughout the reflective-walled cavity 31. When the user is done using the mask 3, in the preferred separable-shell invention embodiment of the invention, the user may separate the outer 15 and inner 23 shell attachments from one another, and thereby separate the inner 2 and outer shells 1 for cleaning.

While it has been illustrated here that the ultraviolet light 13 can be introduced by a light source 12 comprising fiber optics, this is for example, not limitation. The benefit of this configuration is that there is minimal weight added to the overall mask configuration, and there is less heat generated inside the cavity 31, thereby maximizing ergonomic user comfort. This is as opposed to having a complete light source, and possibly even a power source for the light, stationed between the shells 1 and 2. The source of light (not shown) which feeds into the optical fiber can any suitable germicidal ultraviolet light known in the art, and may be powered by batteries or in other equivalent ways, e.g., plugging into an electrical outlet, that are known in the art. A light/battery pack can be carried in user's pocket, attached to a belt, provided with head gear, worn around the neck, or configured in any other manner that is comfortable for the user and does not diminish the introducing of light into the cavity 31. If optical fiber is used for the light source 12, the terminal point of the fiber inside the cavity 31 can be ground into a suitable lensing curvature, or fitted with a light-distributing lens, so as to optimize the distribution of light inside the cavity 31.

The knowledge possessed by someone of ordinary skill in the art at the time of this disclosure, including but not limited to the prior art disclosed with this application, is understood to be part and parcel of this disclosure and is implicitly incorporated by reference herein, even if in the interest of economy express statements about the specific knowledge understood to be possessed by someone of ordinary skill are omitted from this disclosure. While reference may be made in this disclosure to the invention comprising a combination of a plurality of elements, it is also understood that this invention is regarded to comprise combinations which omit or exclude one or more of such elements, even if this omission or exclusion of an element or elements is not expressly stated herein, unless it is expressly stated herein that an element is essential to applicant's combination and cannot be omitted. It is further understood that the related prior art may include elements from which this invention may be distinguished by negative claim limitations, even without any express statement of such negative limitations herein. It is to be understood, between the positive statements of applicant's invention expressly stated herein, and the prior art and knowledge of the prior art by those of ordinary skill which is incorporated herein even if not expressly reproduced here for reasons of economy, that any and all such negative claim limitations supported by the prior art are also considered to be within the scope of this disclosure and its associated claims, even absent any express statement herein about any particular negative claim limitations.

Finally, while only certain preferred features of the invention have been illustrated and described, many modifications, changes and substitutions will occur to those skilled in the art. It is, therefore, to be understood that the appended claims are intended to cover all such modifications and changes as fall within the true spirit of the invention.

I claim:

1. A germicidal facemask apparatus, comprising:
an inner facemask shell shaped to fit in a substantially airtight fashion over a mouth and nose of a human face;
an outer facemask shell shaped to substantially conform to said inner facemask shell;
said inner facemask shell comprising at least one inner shell vent thereof;
said outer facemask shell comprising at least one outer shell vent thereof;
a front surface of said inner facemask shell comprising an inner shell substantially-reflective material thereof;
a rear surface of said outer facemask shell comprising an outer shell substantially-reflective material thereof;
a substantially-airtight attachment of said outer shell with said inner shell about perimeters thereof;
a substantially airtight and light-reflective air cavity between said inner and outer facemask shells, formed by said substantially airtight attachment of said outer shell with said inner shell about said perimeters, and by said inner shell and outer shell substantially-reflective materials; and
an ultraviolet light source configured to introduce ultraviolet light into said air cavity between said inner and outer facemask shells and for said ultraviolet light to substantially permeate said air cavity by virtue of said substantially-reflective materials on said front surface of said inner facemask shell and on said rear surface of said outer facemask shell; wherein
when said germicidal facemask apparatus is placed over a user's face, by virtue of the foregoing configuration combination, air which is breathed in and out by said user will pass through said air cavity while traversing between said at least one inner shell vent and said at least one outer shell vent, and will undergo germicidal sterilization responsive to its being exposed to said ultraviolet light being reflected within said cavity.

2. The apparatus of claim 1, further comprising:
said inner facemask shell being air-impermeable except through said at least one inner shell vent; and
said outer facemask shell being air-impermeable except through said at least one outer shell vent.

3. The apparatus of claim 2, further comprising:
at least one of said outer shell vents aligning with an impermeable portion of said inner shell; and
at least one of said inner shell vents aligning with an impermeable portion of said outer shell; wherein:
said air-impermeability of said shells except through said vents in combination with said vent alignments causes air passing through said cavity to traverse a larger space inside of said cavity before exiting said cavity, thereby prolonging the exposure of the air to said ultraviolet light for germicidal sterilization.

4. The apparatus of claim 1, wherein said substantially-airtight attachment of said outer shell with said inner shell about perimeters thereof is configured to not permit said outer shell and said inner shell to be separated from one another.

5. The apparatus of claim 1, wherein said substantially-airtight attachment of said outer shell with said inner shell about perimeters thereof is configured to permit said outer shell and said inner shell to be separated from one another.

6. The apparatus of claim 1, said substantially-airtight attachment of said outer shell with said inner shell about perimeters thereof comprising a Ziploc®-type seal or equivalent

7. The apparatus of claim 5, said substantially-airtight attachment of said outer shell with said inner shell about perimeters thereof comprising a Ziploc®-type seal or equivalent.

8. The apparatus of claim 1, at least one of said inner facemask shell and said outer facemask shell comprising at least one spacer for maintaining a separation between said inner and outer shells to facilitate breathing airflow and penetration of said ultraviolet light through said air cavity.

9. The apparatus of claim 1, said vents comprising louvers.

10. A method for sterilizing air using a germicidal facemask, comprising:
attaching inner and outer facemask shells of said facemask with one another about perimeters thereof to form a substantially airtight and light-reflective air cavity between said inner and outer facemask shells, said inner facemask shell shaped to fit in a substantially airtight fashion over a mouth and nose of a human face and said outer facemask shell shaped to substantially conform to said inner facemask shell;
introducing ultraviolet light into said air cavity between said inner and outer facemask shells;
substantially permeating said ultraviolet light through said air cavity, using substantially-reflective materials on a front surface of said inner facemask shell and on a rear surface of said outer facemask shell for making said air cavity light-reflective;
placing said germicidal facemask apparatus over a user's face;
the user breathing air in and out through said facemask, wherein said air therefore passes though said air cavity while traversing between at least one inner shell vent of said inner facemask shell and said at least one outer shell vent of said outer facemask shell, said air thereby undergoing germicidal sterilization responsive to its being exposed to said ultraviolet light being reflected within said cavity.

11. The method of claim 10, further comprising:
said inner facemask shell being air-impermeable except through said at least one inner shell vent; and
said outer facemask shell being air-impermeable except through said at least one outer shell vent.

12. The method of claim 11, further comprising causing air passing through said cavity to traverse a larger space inside of said cavity before exiting said cavity, thereby prolonging the exposure of the air to said ultraviolet light for germicidal sterilization, by aligning at least one of said outer shell vents with an impermeable portion of said inner shell, and by aligning at least one of said inner shell vents with an impermeable portion of said outer shell.

13. The method of claim 10, further comprising said substantially-airtight attachment of said outer shell with said inner shell about perimeters thereof configured to not allow separating said outer shell and said inner shell from one another.

14. The method of claim 10, further comprising separating said outer shell and said inner shell from one another along said substantially-airtight attachment of said outer shell with said inner shell about perimeters thereof.

15. The method of claim 10, said attaching said outer shell with said inner shell about perimeters thereof comprising using a substantially-airtight Ziploc®-type seal or equivalent.

16. The method of claim 14, said attaching said outer shell with said inner shell about perimeters thereof comprising using a substantially-airtight Ziploc®-type seal or equivalent.

17. The method of claim 10, further comprising maintaining a separation between said inner and outer shells to facilitate breathing airflow and penetration of said ultraviolet light through said air cavity, using at least one spacer of at least one of said inner facemask shell and said outer facemask shell.

18. The method of claim 10, further comprising louvering said vents.
